(19) United States
(12) Patent Application Publication
(10) Pub. No.: US 2012/0149988 A1
(43) Pub. Date: Jun. 14, 2012

(54) Self-Sealing Surgical Tool

(76) Inventors: Dyson W. Hickingbotham, (US); David E. Booth, (US)

(21) Appl. No.: 13/324,775

(22) Filed: Dec. 13, 2011

Related U.S. Application Data
(60) Provisional application No. 61/422,431, filed on Dec. 13, 2010.

(51) Int. Cl. A61B 1/32

(52) U.S. Cl. 600/208

(57) ABSTRACT

A surgical tool that is self sealing. The surgical tool includes a body and a sealing disc. The body includes a tube positioned at a distal end and a hub at a proximal end. Furthermore, the body includes a sealing disc receiving space positioned through a sidewall of the hub. The sealing disc is securable within the sealing disc receiving space.

### CROSS-REFERENCE TO RELATED APPLICATIONS

This application is claims benefit of the filing date under 35 U.S.C. §119(e) of Provisional Patent Application No. 61/422,431, filed Dec. 13, 2010.

### FIELD OF INVENTION

This invention relates to a surgical tool and in particular to a self-sealing surgical tool.

### BACKGROUND

Generally, a cannula is a tube that can be inserted into the body, often for the delivery or removal of fluid. In medical applications, a cannula can surround the inner or outer surfaces of a Trocar needle, for example, which is passed through the cannula, puncturing the body in order to get into an intended proximate position.

During certain medical procedures, a surgeon may typically use several hand pieces or instruments that are subsequently inserted into and removed from the cannula. This repeated removal and insertion can cause trauma during an ophthalmic procedure, for example. To address this concern, hubbed cannula were developed at least by the mid-1980s. These devices consist of a narrow tube with an attached hub. For example, in an ophthalmic medical procedure, the tube is inserted into an incision in the eye up to its hub, which acts as a stop, preventing the tube from entering the eye completely. Surgical instruments can then be inserted into the eye through the tube, and the tube protects the incision sidewall from repeated contact by the instruments.

The loss of intraocular pressure during an ophthalmic medical procedure is a concern when instruments are exchange or removed through a cannula. The eye, being a pressurized globe, expels aqueous or vitreous out of the open cannula when a surgical device is not present. As a result, seals have been introduced into cannula designs, which self seal upon instrument removal. These cannula, an example of which is shown in US Patent Application Publication Number 2008/0312662, generally consist of a tube and an attached hub, with a sealing disc positioned in an opening that is recessed from a top surface of the hub.

In order to secure the seal in the hub of the cannula, a separate cap is positioned on top of the hub, covering the top surface and the seal positioned in the opening.

There are several disadvantages with these known cannula, which include, inter alia, higher demand in manufacturing the known cannula, which includes several separate pieces notwithstanding the seal. Furthermore, the known cap is a separate piece that sits on top and around the cannula hub, which is not secured to the cannula hub. As a result, the cap and seal maybe become loose during the procedure.

Other cannula arrangements have further disadvantages. The company, DORC has fits a kind of cap over a flange on the hub of the cannula, which cap provides a seal between the instrument and trocar. However, such a cap has the disadvantage of increasing the external diameter of the trocar. Additionally, this method of sealing between the instrument and trocar leaks and is inadequate. The described invention by this application provides a superior sealing arrangement by virtue of the hinge method used to affect the seal.

U.S. Pat. No. 5,865,807 describes a further seal that can be arranged inside a trocar. This seal has two sealing locations. This is advantageous regarding the quality of the seal but disadvantageous in that the instruments have to be pushed longitudinally along the two seals increasing the force necessary to maneuver with in the eye.

### SUMMARY

The present invention improves upon known surgical tool that is self sealing. The self sealing surgical tool is a cannula that includes a body and a sealing disc. The body includes a tube positioned at a distal end and a hub at a proximal end. Furthermore, the body includes a sealing disc receiving space positioned through a sidewall of the hub. The sealing disc is securable within the sealing disc receiving space.

### BRIEF DESCRIPTION OF THE DRAWINGS

The invention will now be described by way of example with reference to the accompanying figures of which:

FIG. 1 is a front view of a cannula with a sealing disc according to the invention;

FIG. 2 is a top view of the cannula of FIG. 1;

FIG. 3 is a cross sectional view of the cannula of FIG. 2, along the line 2-2;

FIG. 4 is a perspective view of a cannula according to the invention;

FIG. 5 is a perspective view of a sealing disc according to the invention;

FIG. 6 is a top view of the sealing disc of FIG. 4;

FIG. 7 is a perspective view of a cannula with an assembled sealing disc according to the invention;

FIG. 8 is a perspective view of another cannula and sealing disc according to the invention;

FIG. 9 is a perspective view of the cannula of FIG. 8;

FIG. 9 is a perspective view of the sealing disc of FIG. 8;

FIG. 11 is a perspective view of another cannula and sealing disc according to the invention; and

FIG. 12 is a perspective view of the cannula of FIG. 11.

### DETAILED DESCRIPTION OF THE EMBODIMENT(S)

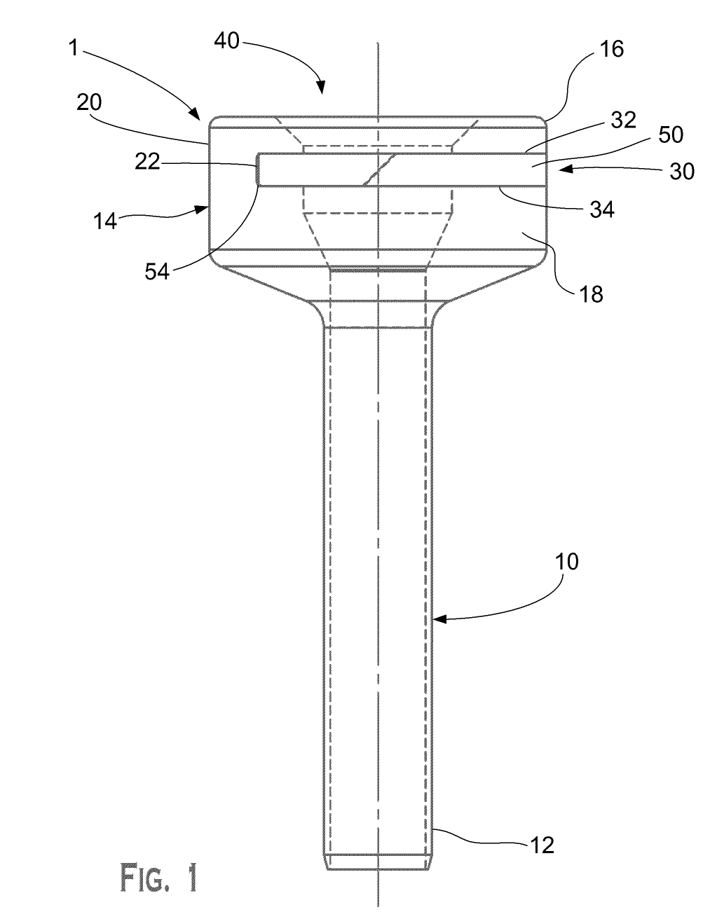

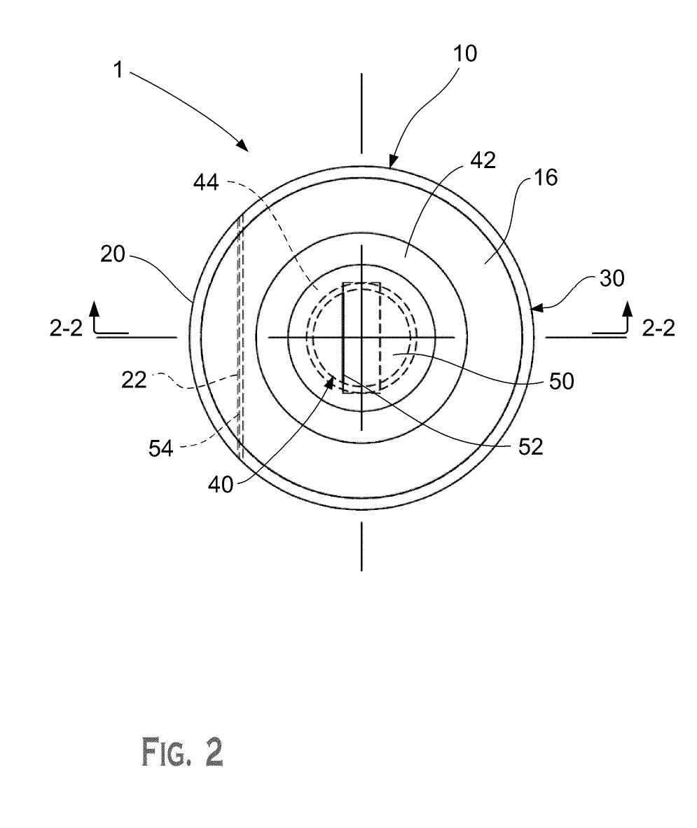

The invention will now be described in greater detail first with reference to FIGS. 1-7.

With respect to FIGS. 1-4, a cannula 1 according to the invention is shown having a body 10 and sealing disc 50, wherein the sealing disc 50 fits securely through a sidewall of the body 10.

The body 10 has a tube 12 at a distal end, and a hub 14 at a proximal end. The hub 14 is positioned on top of the tube 12, and includes a sealing disc receiving space 30 passing through a portion of a sidewall of the hub 14. The hub 14 and sealing disc receiving space 30 are designed such that the sealing disc 50 may be fittingly secured within the hub 14, which will be discussed in more detail below.

The body 10 may be made from any material suitable for medical procedures, such as stainless steel, titanium, or thermoplastic, while the sealing disc 50 may be made form an elastically deformable material, such as a thermoplastic.

The tube 12 is a hollow, elongated structure, extending from a bottom surface of the hub 14. The tube 12 has a narrow external tubular wall which tapers toward a distal end of the cannula 1. The tube 12 is wide enough such that medical instruments can enter and extend through the tube 12.

The hub 14 is wider than the tube 12, and has a cylindrical shape in the embodiment shown. The hub 14 has an opening 40 formed along a top surface of the hub 14. The opening 40 extends through the hub 14 and into tube 12. The opening 40 and the tube 12 are aligned, such that a medical instrument can enter into the opening 40 and extend through the hollow tube 12.

The hub 14 is a monolithic structure having a cover 16 and a base 18, which are connected together by a bridge 20. The sealing disc receiving space 30 is provided between the base 18 and the cover 16, and closed at one end by the bridge 20. It also possible that the sealing disc receiving space 30 is closed along multiple sidewalls limiting the openings available for ingress and egress of the sealing disc 50. In the shown embodiment, the hub 14 is cylindrical; however, other polygonal shapes are possible, which may include multiple sidewalls.

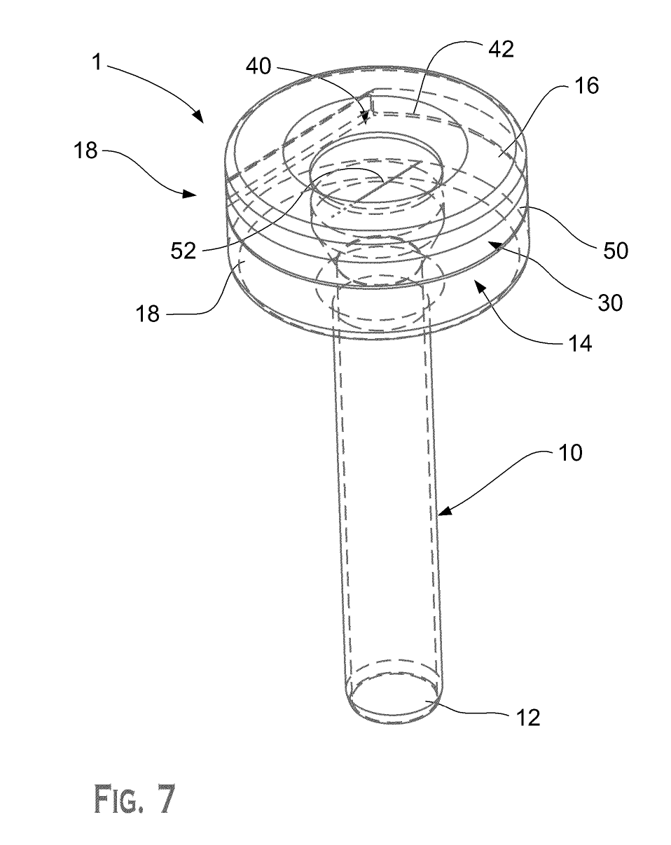

The cover 16 is generally the top section of the hub 14, while the base 18 is the bottom section of the hub 14. The bridge 20 is provided when the sealing disc receiving space 30 is formed. In the embodiment shown, the sealing disc receiving space 30 is formed in the hub 14 by cutting out a proximate middle section of the hub 14 from a single side. As shown in the FIG. 6, the hub 14 according to the invention is cylindrical, as is the sealing disc receiving space 30. The sidewall surfaces and internal body of the hub 14 have been milled, except for a section of the hub 14, which makes up the bridge 20. Accordingly, the sealing disc receiving space 30 is a slot that is accessible from the sidewall of the hub 14, and has a height H_{c} that is defined by the space between a ceiling 32 and floor 34. The ceiling 32 is a bottom surface of the cover 16, while the floor 34 is a top surface of the base 18.

Since the bridge 20 connects the cover 16 and the base 18, the bridge 20 provides structural integrity to the cover 16 and base 18, It should be understood that the bridge 20 is not limited to the shape or size shown in this embodiment, and is intended to support the cover 16 in such a way that the cover 16 is not damaged by external forces. Furthermore, the bridge 20 is a wall and guide for an incoming sealing disc 50, being positioned in the sealing disc receiving space 30. The bridge 20 includes a key 22 which is a flat surface in the embodiment shown. This key 22 cooperates with the sealing disc 50 to facilitate it's alignment within the sealing disc receiving space 30 in an operating position. Although the key 22 is flat in the embodiment shown, the key 22 could include any number of teeth or shapes that would match corresponding teeth and shapes on a receiving sealing disc 50. In fact, the key 22 can be angled, as well. An outer edge of the key 22 may extend slightly back toward the bridge 20 and the base 18, such that the angled key 22, when matched with a corresponding angled disc key 54, provides further alignment and securing of the sealing disc 50 within the hub 14. Depending on the material properties, the bridge 20 may be elastically deformable, and can deform in such a way that the cover 16 can rotate away from the base 18, or toward the base 18, as to close a sealing disc 50 within the sealing disc receiving space 30.

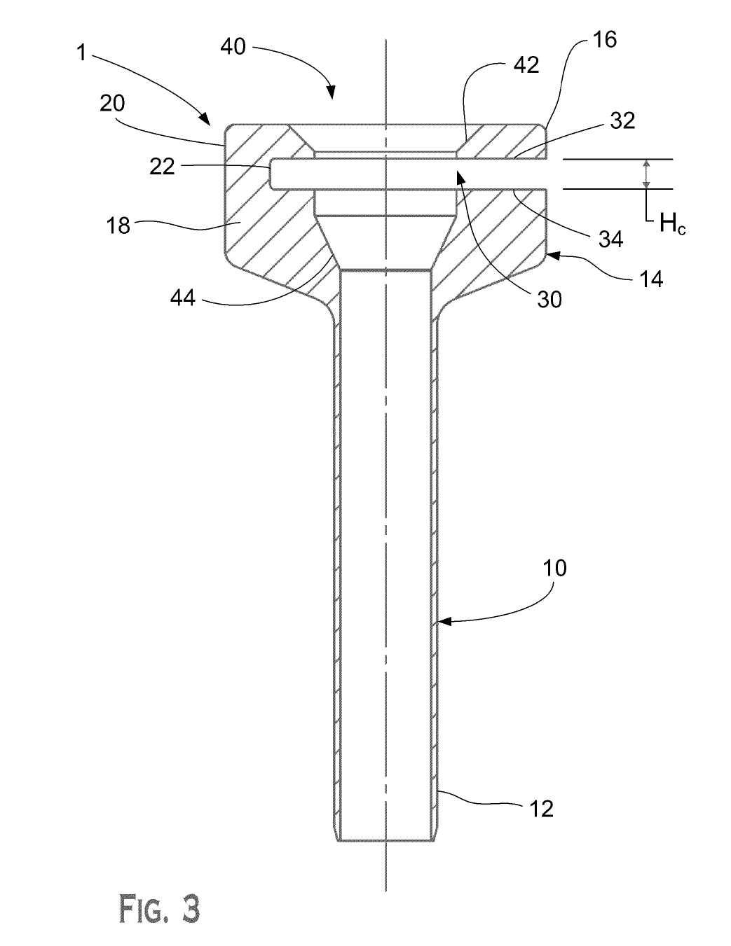

The opening 40 includes an external receiving passageway 42 provided through the cover 16 and an internal receiving passageway 44 through the base 18 of the hub 14 (see FIG. 3). The external receiving passageway 42 is a defined opening in a top surface in the cover 16, which is circular in the embodiment shown. The external receiving passageway 42 then slopes or tapers in such a way to have a funnel shape directed into the sealing disc receiving space 30, and then into the internal receiving passageway 44. The internal receiving passageway 44 is a defined opening in a top surface the base 18, which also slopes or tapers in such a way to have a funnel shape. However, the internal receiving passageway 44 is directed into the hollow tube 12.

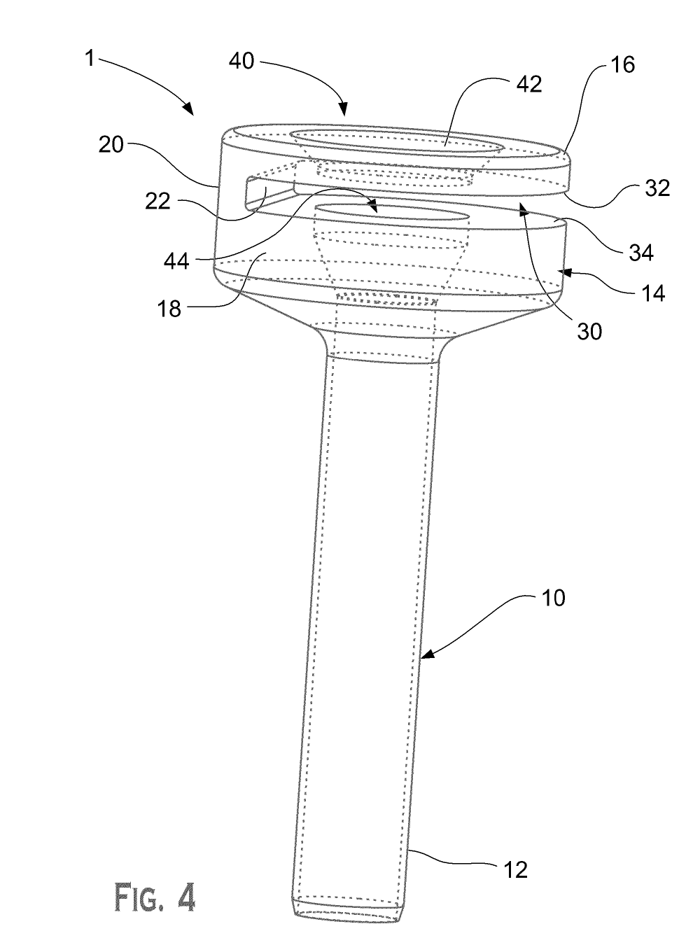

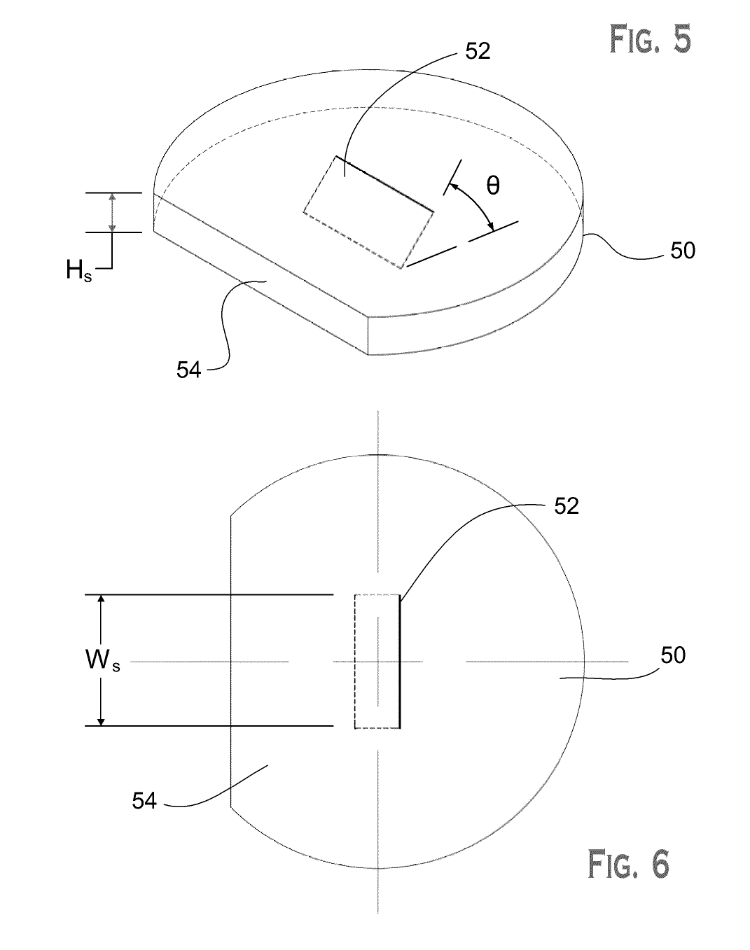

With reference to FIGS. 4 and 5, a sealing disc 50 according the invention is shown. The sealing disc 50 is roughly circular, and is sized and shaped to fit within sealing disc receiving space 30 of the hub 14. The sealing disc 50 includes a slit 52 and a disc key 54 that corresponds to the key 22 of the bridge 20. The height Hₛ of the sealing disc 50 (FIG. 5) is equal to or less than the height H_{c} of the sealing disc receiving space 30 (FIG. 3). However, if the bridge 20 is elastically deformable, and depending on the elasticity of the sealing disc 50, the height Hₛ can be slightly larger than the height H_{c} of the sealing disc receiving space 30, such that that the sealing disc 50 may fit snuggly in the sealing disc receiving space 30.

The slit 52 is positioned at a proximate middle of the sealing disc 50, which is in alignment with the opening 40, external receiving passageway 42, internal receiving passageway 44, and opening of the hollow tube 12. The slit 52 extends through the thickness of sealing disc 50, and is angled between about 40-50 degrees (most preferably 45 degrees), but it should be understood that other angles are within the scope and spirit of the invention and so the angle may be varied depending upon the requirements of a particular application.

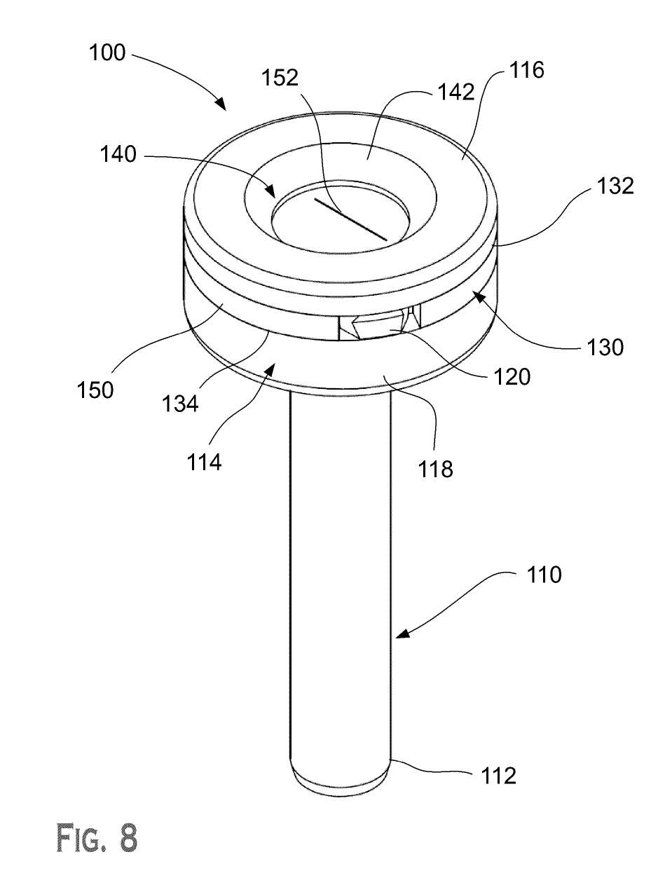

Referring to FIG. 7, an assembly of the cannula 1 is shown, with the sealing disc 50 positioned in the hub 14. The sealing disc 50 slides into the sealing disc receiving space 30 of the hub 14, and is seated on the top surface of the base 18. The key 22 of the bridge 20 comes into contact with the disc key 54 of the sealing disc 50, which positions the sealing disc 50 in an operating position. The slit 52 of the sealing disc 50 is aligned with the opening 40 and hollow tube 12, such that an instrument can enter through the external receiving passageway 42 in the cover 16 and into the slit 52. The instrument self seals as it extends through the slit 52 and then through the internal receiving passageway 44 of the base 18. Since the hollow tube 12 is aligned with the opening 40 of the hub 14, the instrument can further extend into and through the hollow tube 12. When the instrument is pulled out through of the cannula 1, the sealing disc 50 seal seals the internal contents of the body 10, including the hollow tube 12 and the internal receiving passageway 44 of the hub 14. Advantageously, the inventive cannula 1 does not require a separate cap that fits over the hub 14, which can become displaced during operation and compromise the sealing properties of the cannula 1.

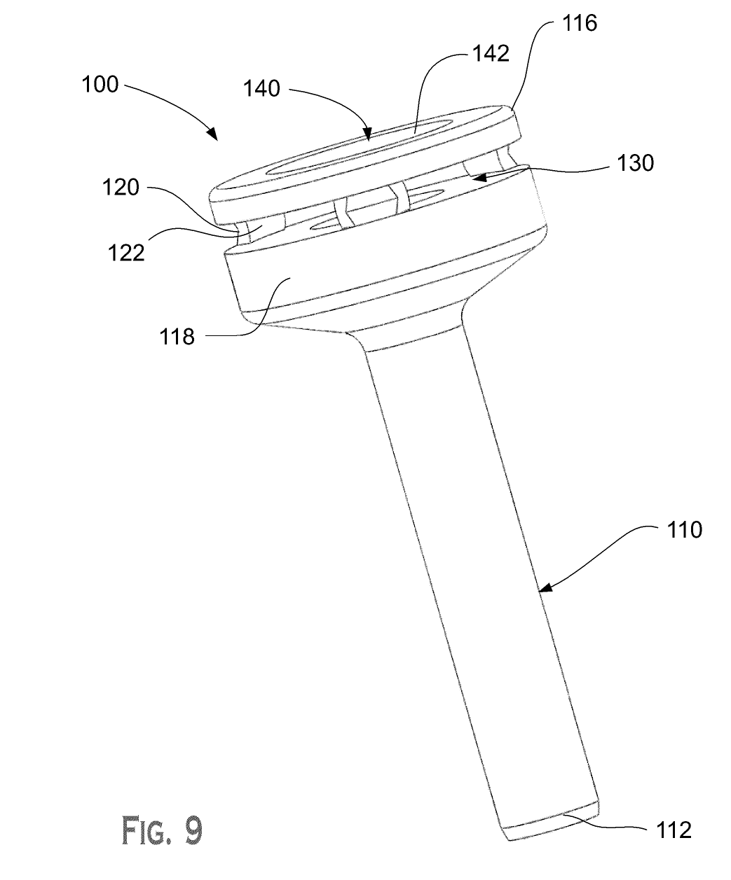

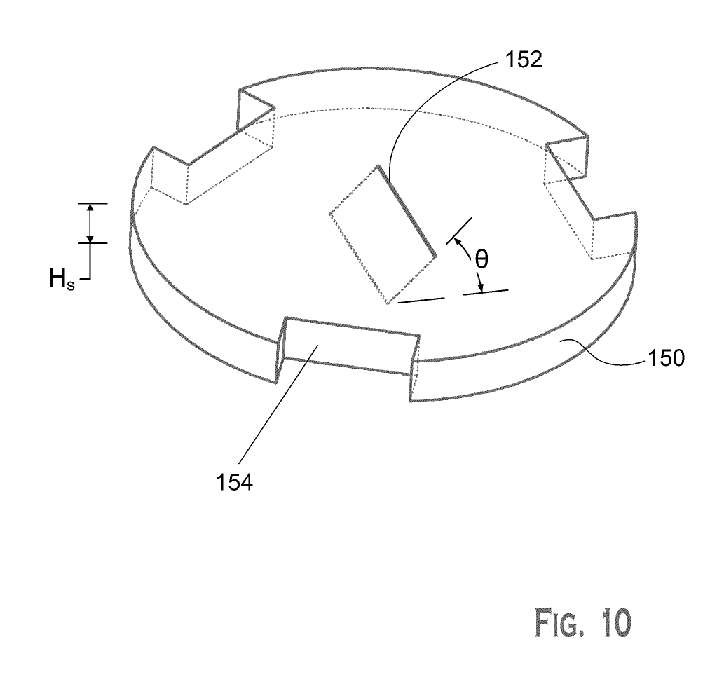

With reference to FIGS. 8 through 10, another cannula 100 according to the invention is shown, having body 110 and sealing disc 150, wherein the sealing disc 150 fits securely through a sidewall of the body 110.

The body 110 has a tube 112 at a distal end, and a hub 114 at a proximal end. The hub 114 is positioned on top of the tube 112, and includes a sealing disc receiving space 130 passing through a portion of a sidewall of the hub 114. The hub 114 and sealing disc receiving space 130 are designed such that the sealing disc 150 may be fittingly secured within the hub 14.

Notably, the cannula 100 includes a plurality of collapsible bridges 120. The collapsible bridges 120 connect the cover 116 and the base 118, and include perforations 122, which assist in collapse of the collapsible bridge 120 when pressure is applied to the hub 114.

The sealing disc 150 includes a number of notches 154, as shown in FIG. 10. The notches 154 receive the collapsible bridges 120. The sealing disc receiving space 130 has enough height clearance such that the sealing disc 150 can be maneuvered into position, such that the sealing disc 150 lays flat on the base 118 and the collapsible bridges 120 are received by the notches 154. A force can be applied to the cover 116, collapsing the cover 116 on the sealing disc 150 in the sealing disc receiving space 130. As a result, the sealing disc 150 is ten pinned between the cover 116 and the base 118.

Advantageously, the inventive cannula 100 does not require a separate cap that fits over the hub 114, which can become displaced during operation and compromise the sealing properties of the cannula 100.

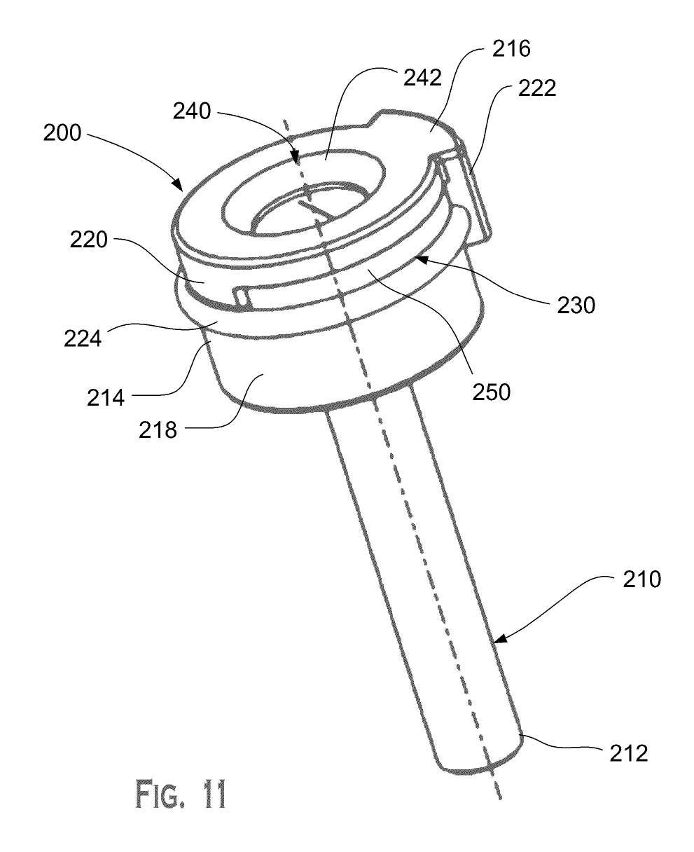

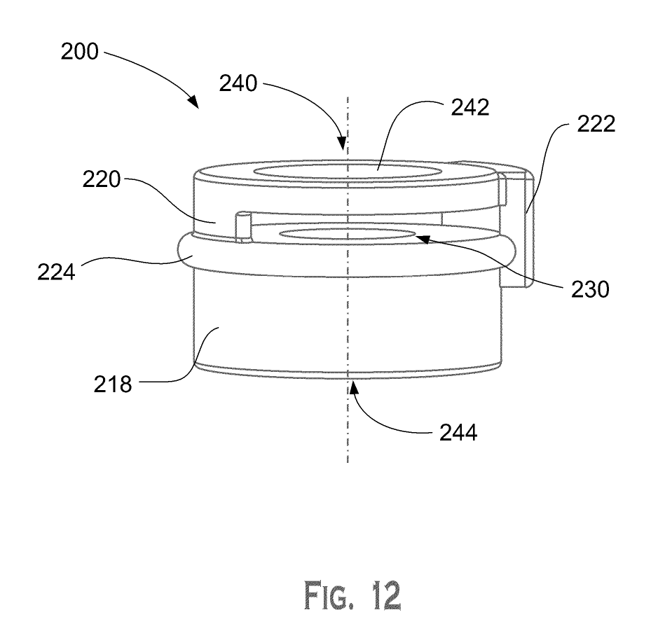

With respect to FIGS. 11 and 12, another cannula 200 according to the invention is shown having a body 210 and sealing disc 250, wherein the sealing disc 250 fits securely through a sidewall of the body 210.

The body 210 has a tube 212 at a distal end, and a separable hub 214 at a proximal end. The hub 214 is positioned on top of the tube 212, and includes a sealing disc receiving space 230 passing through a portion of a sidewall of the hub 214. The hub 214 and sealing disc receiving space 230 are designed such that the sealing disc 250 may be fittingly secured within the hub 214.

The tube 212 is a hollow, elongated structure, and extends into a bottom surface of the hub 214, in the embodiment shown. The tube 212 has a narrow external tubular wall which tapers toward a distal end of the cannula 1. The tube 212 is wide enough such that medical instruments can enter and extend through the tube 212

The hub 214 is wider than the tube 212, and has a cylindrical shape in the embodiment shown. The hub 214 has an opening 240 formed along a top surface of the hub 214. The opening 240 extends through the hub 214 and into tube 212. The opening 240 and the tube 212 are aligned, such that a medical instrument can enter into the opening 240 and extend through the hollow tube 212.

The hub 214 is a monolithic structure having a cover 216 and a base 218, which are connected together by a bridge 220. The sealing disc receiving space 230 is provided between the base 218 and the cover 216, and closed at one end by the bridge 220 and a latch 222 at an opposite end in the embodiment shown. It also possible that the sealing disc receiving space 230 is closed along multiple sidewalls limiting the openings available for ingress and egress of the sealing disc 250.

The cover 216 is generally the top section of the hub 214, while the base 218 is the bottom section of the hub 214. In the embodiment shown, the sealing disc receiving space 230 is formed in the hub 214 by cutting out a proximate middle section of the hub 214. As shown in the FIG. 11, the hub 214 is generally cylindrical, as is the sealing disc receiving space 30.

However, the latch 222 projects out from the outer surface of hub 214. Additionally, the hub 214 includes a circumferential projection 224. However, while it is shown that the circumferential projection 224 encompasses the outer surface of the hub 214, the circumferential projection 224 may be positioned as to connect with the latch 222.

Since the bridge 220 connects the cover 216 and the base 218, the bridge 220 provides structural integrity to the cover 216 and base 218, yet is elastically deformable such that the cover 216 can rotate away from the base 218. The bridge 20 is also a wall and/or guide for the incoming sealing disc 250, which is positioned in the sealing disc receiving space 30 between the cover 216 and the base 218. The bridge 220 may include a key (not shown), as discussed above.

The bridge 220, being elastically deformable, and can deform in such a way that the cover 16 can rotate away from the base 18, or toward the base 18. When the bridge 220 deforms such that the cover 216 rotates toward the base 218, the cover 216 close the sealing disc 250 within the sealing disc receiving space 230. The latch 222 connects with the circumferential projection 224, and holds the cover 216, pinning the sealing disc 250 between the cover 216 and the base 218.

As shown in FIG. 12, the hub 214 is separable from the tube 212. This allows the hub 214 to be manufactured from a material different than the tube 212. In the embodiment shown, the tube 212 connects with the internal receiving passageway 244 of the hub 214. The connection is not permanent, but rather a temporary connection known to one skilled in the art.

Advantageously, the inventive cannula 200 does not require a separate cap that fits over the hub 214, which can become displaced during operation and compromise the sealing properties of the cannula 200.

While certain embodiments of the present invention have been described above, these descriptions are given for purposes of illustration and explanation. Variations, changes, modifications and departures from the systems and methods disclosed above may be adopted without departure from the scope or spirit of the present invention.

What is claimed is:

1. A surgical tool, comprising:
a body having a tube at a distal end, a hub at a proximal end, and a sealing disc receiving space positioned through a sidewall of the hub; and
a sealing disc that is securable within the sealing disc receiving space.

2. The surgical tool according to claim 1, wherein the sealing disc receiving space passes through a portion of the sidewall.

3. The surgical tool according to claim 1, wherein the hub is wider than the tube, and has an opening formed along a top surface of the hub.

4. The surgical tool according to claim 3, wherein the opening extends through the hub and into the tube.

5. The surgical tool according to claim 1, wherein the hub is a monolithic structure having a cover and a base that are connected together by a bridge.

6. The surgical tool according to claim 5, wherein the sealing disc receiving space is positioned between the base and the cover, and closed along at least one end by the bridge.

7. The surgical tool according to claim 5, wherein the sealing disc receiving space is formed in the hub by cutting out a proximate middle section of the hub from a side.

8. The surgical tool according to claim 5, wherein the bridge is a wall and guide for the sealing disc.

9. The surgical tool according to claim 5, wherein the bridge includes a key which is an abutting surface for the sealing disc to facilitate alignment within the sealing disc receiving space in an operating position.

10. The surgical tool according to claim 9, wherein the key includes a key contour that corresponds to a disc contour on a disc key of the sealing disc.

11. The surgical tool according to claim 5, wherein the bridge is elastically deformable in such a way that the cover is movable away from or toward the base, to place and secure the sealing disc within the sealing disc receiving space respectively.

12. The surgical tool according to claim 5, wherein the sealing disc receiving space is a slot that is accessible from the sidewall of the hub, and has a height Hc that is defined by a space between a bottom surface of the cover and a top surface of the base.

13. The surgical tool according to claim 12, wherein a height Hs of the sealing disc is equal to or less than the height Hc of the sealing disc receiving space.

14. The surgical tool according to claim 12, wherein the bridge is elastically deformable in such a way that the cover is movable away from or toward the base, to place and secure the sealing disc within the sealing disc receiving space respectively.

15. The surgical tool according to claim 12, wherein a height Hs of the sealing disc is larger than the height Hc of the sealing disc receiving space.

16. The surgical tool according to claim 1, wherein the sealing disc slides into the sealing disc receiving space of the hub.

17. The surgical tool according to claim 1, wherein the hub is a monolithic structure having a cover and a base that are connected together by at least two collapsible bridges.

18. The surgical tool according to claim 17, wherein each collapsible bridge is elastically deformable such that the cover is movable away from or toward the base, to place and secure the sealing disc within the sealing disc receiving space respectively.

19. The surgical tool according to claim 18, wherein each collapsible bridge includes perforations.

20. The surgical tool according to claim 1, wherein the sealing disc includes a number of notches to receive corresponding collapsible bridges.

21. A surgical tool, comprising:
an elongated hollow tube;
a monolithic hub positioned above the elongated hollow tube and having a cover and a base that are connected together by a deformable bridge, the cover having an opening that aligns with the elongated hollow tube; and
a sealing disc that is securable within a sealing disc receiving space between the cover and the base.

22. The surgical tool according to claim 21, wherein the opening extends through the monolithic hub and into the elongated hollow.

23. The surgical tool according to claim 21, further comprising a latch at an opposite end from the deformable bridge.

24. The surgical tool according to claim 23, wherein the latch projects out from an outer surface of the cover.

25. The surgical tool according to claim 24, wherein the base includes a circumferential projection that encompasses the outer surface of the base and is positioned to connect with the latch.

26. The surgical tool according to claim 25, wherein the latch engages the circumferential projection.

27. The surgical tool according to claim 21, wherein the deformable bridge is movable away from or toward the base, to place and secure the sealing disc within the sealing disc receiving space respectively.

28. The surgical tool according to claim 21, wherein the deformable bridge includes a key that corresponds with a disc key on the sealing disc.

29. The surgical tool according to claim 21, wherein the monolithic hub is separable from the elongated hollow tube.
